(19) United States
(12) Patent Application Publication
(10) Pub. No.: US 2026/0355107 A1
(43) Pub. Date: Nov. 12, 2026

(54) METHOD TO PREPARE MULTILAYERED DRUG DELIVERY DEVICE

(71) Applicant: Sriyan Daggubati, Danville, CA (US)

(72) Inventor: Sriyan Daggubati, Danville, CA (US)

(21) Appl. No.: 19/671,316

(22) Filed: May 8, 2026

Related U.S. Application Data
(60) Provisional application No. 63/802,620, filed on May 9, 2025.

(51) Int. Cl. A61K 9/00; A61K 9/70; A61K 47/34; A61K 47/42

(52) U.S. Cl. CPC A61K 9/0024; A61K 9/7007; A61K 47/34; A61K 47/42

(57) ABSTRACT

A method to prepare a multilayered biodegradable drug delivery device is disclosed. At one step, a plurality of poly(lactic-co-glycolic acid) (PLGA) films is provided. At another step, each PLGA film is coated with a gelatin solution to obtain gelatin-coated PLGA films. At another step, the gelatin-coated PLGA films undergo a crosslinking process using a crosslinking agent. At another step, a specialized pro-resolving mediator (SPM) is loaded between the gelatin-coated PLGA films. At another step, the SPM-loaded gelatin-coated PLGA films are assembled into a multilayered structure to obtain the multilayered biodegradable drug delivery device. The SPM comprises a Resolvin D1 (RvD1) solution. The use of differing layer compositions might generate asymmetric hydration and degradation behavior across the multilayer structure that allow sustained, directional release of RvD1 toward the vascular injury site or tissue-facing surface.

### BACKGROUND

### Technical Field

The subject matter relates to a device that is utilized for drug delivery and methods of making the devices. More particularly, the subject matter relates to the preparation of multilayered biodegradable thin films designed for the controlled release of resolvins, particularly Resolvin D1 (RvD1), at vascular injury sites to facilitate localized inflammation resolution and tissue regeneration.

### Discussion of the Related Art

Vascular injuries and diseases often lead to persistent inflammation, hindering natural healing and increasing the risk of complications like graft failure and thrombosis. Specialized pro-resolving mediators (SPMs), including Resolvin D1 (RvD1), derived from omega-3 fatty acids, offer a promising therapeutic avenue by actively promoting the resolution of inflammation and tissue repair. RvD1, in particular, exhibits potent anti-inflammatory and pro-resolving properties by modulating immune cell activity and enhancing the clearance of cellular debris.

Current systemic delivery methods for specialized pro-resolving mediators (SPMs), such as intravenous, subcutaneous, or intraperitoneal administration, are limited by poor tissue specificity and low bioavailability. These approaches often result in widespread distribution of the therapeutic agent, thereby reducing its efficacy at localized sites of vascular injury. Additionally, the systemic delivery methods often result in poor targeting and limited bioavailability. Further, the clinical translation of RvD1 is hampered by its rapid degradation and the difficulty in achieving sustained, localized delivery to the injured vasculature, limiting its therapeutic efficacy.

However, existing delivery systems for delivering SPMs often lead to rapid diffusion away from the target inflammation site, resulting in insufficient local drug concentration and reduced therapeutic efficacy. Further, the RvD1 has a short biological half-life, necessitating repeated administration to maintain effective levels. Conventional delivery systems further lack directional release toward the vessel wall, leading to inefficient targeting of the affected vascular tissue.

Therefore, there is a need for advanced drug delivery devices capable of targeting RvD1 directly to injured vascular tissue in a sustained and controlled manner, thereby enhancing therapeutic efficacy while minimizing off-target effects on surrounding healthy tissues.

### SUMMARY

The proposed technology discloses a method of preparing a multilayered biodegradable drug delivery device. At one step, a plurality of poly(lactic-co-glycolic acid) (PLGA) films is provided. At another step, each PLGA film is coated with a gelatin solution to obtain gelatin-coated PLGA films. At another step, the gelatin-coated PLGA films undergo a crosslinking process using a crosslinking agent. At another step, a specialized pro-resolving mediator (SPM) is loaded between the gelatin-coated PLGA films. At another step, the SPM-loaded gelatin-coated PLGA films are assembled into a multilayered structure to obtain the multilayered biodegradable drug delivery device.

In an embodiment, a structural assembly of the multilayered biodegradable drug delivery device might be a tri-layered structure. The multilayered biodegradable drug delivery device comprises a first layer, a second layer, and an intermediate layer disposed between the first layer and the second layer. Further, the multilayered biodegradable drug delivery device comprises a bioactive agent loaded between the first layer and the intermediate layer, and between the intermediate layer and the second layer. The bioactive agent is a specialized pro-resolving mediator (SPM). Each of the first layer, the intermediate layer, and the second layer comprises a gelatin-coated PLGA film having a distinct relative proportion to facilitate directional release of the SPM from the drug delivery device to a targeted site. The targeted site is a site of a vascular condition selected from the group consisting of vascular injury, vascular inflammation, ischemia-reperfusion injury, endotoxin-induced vascular damage, vascular trauma, graft failure, and thrombosis.

### BRIEF DESCRIPTION OF DRAWINGS

Exemplary embodiments of the present invention will be understood and appreciated more fully from the following detailed description, taken in conjunction with the drawings in which,

FIG. 1 illustrates a method 100 to prepare a Poly(lactic-co-glycolic acid) (PLGA) film, in accordance with an embodiment;

FIG. 2 illustrates a method 200 to prepare a drug delivery device of the present invention, in accordance with an embodiment;

FIG. 3A shows the effect of the RvD1 drug-loaded multilayered biodegradable drug delivery device on TNF-α-induced nuclear factor-κB (NF-κB) activation in vascular smooth muscle cells, in accordance with an embodiment;

FIG. 3B shows the effect of the RvD1-loaded multilayered biodegradable drug delivery device on vascular smooth muscle cell proliferation, in accordance with an embodiment;

FIG. 3C shows the effect of the RvD1-loaded multilayered biodegradable drug delivery device on vascular smooth muscle cell migration, in accordance with an embodiment; and

FIG. 4 illustrates a structural assembly of the drug delivery device, in accordance with another embodiment.

### DETAILED DESCRIPTION

The following detailed description includes references to the accompanying drawings, which form part of the detailed description. The drawings show illustrations in accordance with example embodiments. The numerals in the figure represent like elements throughout the several views, exemplary embodiments of the present disclosure are described. For convenience, only some elements of the same group might be labelled with numerals. The purpose of the drawings is to describe exemplary embodiments and not for production. Therefore, features shown in the figures are chosen for convenience and clarity of presentation only. Moreover, the language used in this disclosure has been principally selected for readability and instructional purposes and might not have been selected to delineate or circumscribe the inventive subject matter, resort to the claims being necessary to determine such inventive subject matter. These example embodiments are described in enough detail to enable those skilled in the art to practice the present subject matter. However, it will be apparent to a person of ordinary skill in the art that the present invention might be practiced without these specific details. The embodiments can be combined, other embodiments can be utilized, or structural and logical changes can be made without departing from the scope of the invention. The following detailed description is, therefore, not to be taken in a limiting sense.

In this document, the terms “a” or “an” are used, as is common in patent documents, to include one or more than one. In this document, the term “or” is used to refer to a non-exclusive “or” such that “a or b” includes “a but not b,” “b but not a,” and “a and b,” unless otherwise indicated.

Reference in the specification to “one embodiment” or to “an embodiment” means that a particular feature, structure, or characteristic described in connection with the embodiments is included in at least one embodiment of the invention, and multiple references to “one embodiment” or “an embodiment” should not be understood as necessarily all referring to the same embodiment.

The proposed technology relates to a preparation of a drug delivery device. The drug delivery device of the proposed technology is a therapeutic film which is biodegradable and gelatin based. The drug delivery device is designed for the controlled and sustained release of drug.

In an embodiment, the drug delivery device is a multilayered biodegradable film. In an embodiment, the multilayered biodegradable film could be a tri-layered structure.

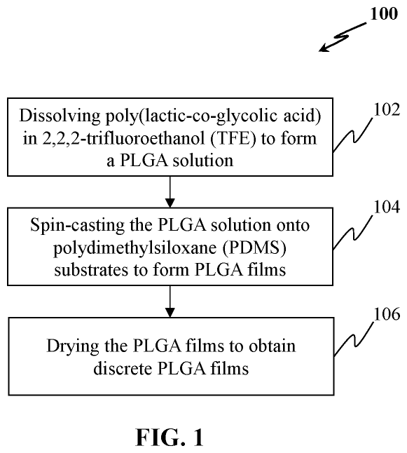

The preparation of the multilayered biodegradable drug delivery device involves the preparation of a poly(lactic-co-glycolic acid) (PLGA) film. Referring to FIG. 1, a method 100 for preparing poly(lactic-co-glycolic acid) (PLGA) films is disclosed. At step 102, the poly(lactic-co-glycolic acid) is dissolved in 2,2,2-trifluoroethanol (TFE) to obtain a PLGA solution. In an embodiment, the PLGA solution is prepared at a concentration of 50 mg/mL to about 500 mg/mL in 2,2,2-trifluoroethanol. In the preferred embodiment, the PLGA solution is prepared at a concentration of 300 mg/mL in 2,2,2-trifluoroethanol. In one embodiment, the lower concentrations might produce thinner and more flexible films, while higher concentrations might produce thicker films with slower degradation and reduced permeability. The concentration disclosed is for exemplary purpose and might vary, that is to say the concentration disclosed is not limited to the specified range.

At step 104, the PLGA solution is spin-casted onto polydimethylsiloxane (PDMS) substrates to form PLGA films. In an embodiment, the spin-casting process might be done by using a spin coater. The spin-casting includes a two-step preparation. The first step of spin-casting includes spinning the PDMS substrates at a first speed of about 1000 rpm for about 30 seconds. The second step of spin-casting includes increasing the spinning speed to a second speed of about 2000 rpm for an additional 30 seconds to achieve a uniform film thickness. In one embodiment, the dried PLGA film might be removed from the PDMS substrate by the manual edge-lifting method and peeling method because PDMS has a low-surface-energy surface that allows the PLGA film to separate after solvent evaporation. In one embodiment, a corner or edge of the dried PLGA film is gently lifted using forceps or a thin blade, then peeled away from the PDMS substrate. In one embodiment, the PDMS substrate might be slightly flexed to initiate separation. If needed, a small amount of sterile water, PBS, or aqueous buffer might be introduced at the film edge to assist release, but full immersion is not required in the preferred process.

At step 106, the PLGA films are dried to obtain discrete PLGA films. In an embodiment, the PLGA film might be substantially dried to remove residual TFE before gelatin coating. Further, the PLGA film might be dried at a temperature in the range of 20 °C to about 40 °C for about 30 minutes to about 24 hours. The drying process might include, but not limited to, ambient air, a desiccator, vacuum drying, or a fume hood. In the preferred embodiment, the PLGA film is dried at room temperature, about 20 °C to 25 °C, under ambient or desiccated conditions for about 12 to 24 hours, or until residual solvent is substantially removed. The PLGA surface might be free of visible solvent, bubbles, cracks, or nonuniform regions.

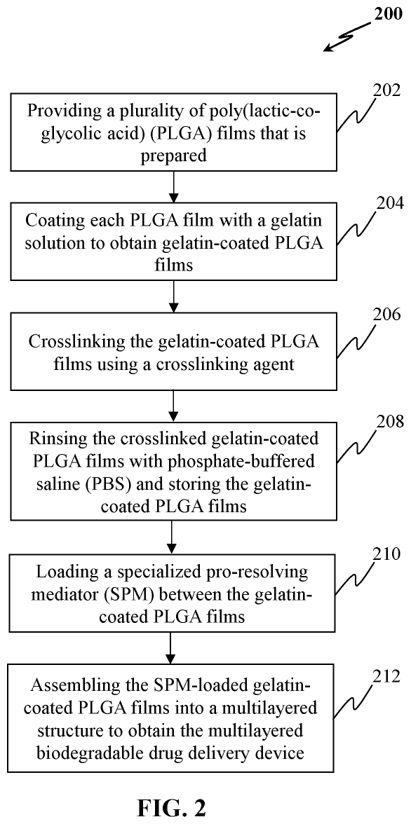

Referring to FIG. 2, a method 200 of preparing a multilayered biodegradable drug delivery device is disclosed. At step 202, a plurality of poly(lactic-co-glycolic acid) (PLGA) films prepared according to the method 100 are provided.

At step 204, each PLGA film is coated with a gelatin solution to obtain gelatin-coated PLGA films. In an embodiment, the gelatin solution is prepared by dissolving a gelatin powder in an aqueous medium at a temperature in the range of about 30 °C to about 40 °C under continuous stirring at about 200 rpm to about 400 rpm for about 30 minutes to about 90 minutes to form the gelatin solution. In an embodiment, the aqueous medium might include, but not limited to, distilled water or a phosphate-buffered saline (PBS) solution. In the preferred embodiment, the temperature is about 37 °C. In an embodiment, the concentration of gelatin in the gelatin solution ranges from about 1% (w/v) to about 5% (w/v). In preferred embodiment, the concentration of gelatin in the gelatin solution is about 2% (w/v). The gelatin solution is followed by degassing under vacuum for about 3 minutes to about 10 minutes. In the preferred embodiment, the degassing of the gelatin solution is performed for about 5 minutes before coating. The degassing step is critical for the elimination of entrapped air bubbles, which, if present, could adversely affect the uniformity and mechanical integrity of the gelatin-coated PLGA films. In one embodiment, the gelatin solution comprises a curing agent that improve the toughness and tensile strength of the layer. In an embodiment, the curing agent might be sulphonyl or aldehydes.

In an embodiment, the gelatin solution is adjusted to a pH in the range of about 4.0 to 9.0. In another embodiment, the gelatin solution is adjusted to the pH in the range of about 6.5 to 7.8. In the preferred embodiment, the pH might be adjusted to 7.2 to 7.4. In an embodiment, the pH is adjusted using a buffer. The buffer might include, but not limited to,

### Phosphate or Acetate Buffer.

In an embodiment, the gelatin solution might be coated using a casting method, a manual spreading method, an immersion coating, a spray coating method or a dip coating method. In an embodiment, the parameters during the gelatin coating process are as provided below. In the preferred embodiment, the gelatin solution might be coated using the dip coating method or immersion coating method. In an embodiment, the immersion coating process might be carried out at a temperature in the range of about 25 °C to about 45 °C. In another embodiment, the immersion coating process might be carried out at a temperature in the range of about 30 °C to about 40 °C. In the preferred embodiment, the immersion coating process might be carried out at a temperature of about 37 °C. The gelatin solution might remain sufficiently fluid near 37 °C, thereby improving coating uniformity while avoiding harsh thermal conditions that could adversely affect the film structure. In an embodiment, the gelatin coating process might be carried out for a duration of about 10 seconds to about 30 minutes. In another embodiment, the gelatin coating process might be carried out for a duration of about 30 seconds to about 10 minutes. In the preferred embodiment, the gelatin coating process might be carried out for a duration of about 1 minute to about 3 minutes.

In an embodiment, each PLGA film is immersed in the degassed gelatin solution maintained at a temperature of about 37 °C for about 30 seconds to about 10 minutes, preferably about 1 to 3 minutes, then withdrawn at a controlled rate to form a uniform gelatin coating. In an embodiment, the gelatin coating has a thickness of about 1 μm to about 100 μm on each PLGA film. In another embodiment, the gelatin coating has a thickness of about 5 μm to about 50 μm on each PLGA film. In another embodiment, the gelatin coating has a thickness of about 5 μm to about 50 μm on each PLGA film. In a preferred embodiment, the gelatin coating has a thickness of about 10 μm on each PLGA film.

Subsequently, the gelatin-coated PLGA film might be dried under controlled temperature and humidity to prevent cracking, uneven shrinkage, or excessive swelling. In an embodiment, the parameters during the drying process of the gelatin-coated PLGA film are as provided below. In an embodiment, the gelatin-coated PLGA film might be dried at a temperature in the range of about 15 °C to about 40 °C. In a preferred embodiment, the gelatin-coated PLGA film might be dried at a temperature in the range of about 20 °C to about 25 °C. In an embodiment, the drying process might be carried out at a relative humidity in the range of about 20% to about 70% RH. In a preferred embodiment, the drying process might be carried out at a relative humidity in the range of about 30% to about 50% RH.

In an embodiment, the drying process might be carried out for a duration of about 10 minutes to about 24 hours. In a preferred embodiment, the drying process might be carried out for a duration of about 30 minutes to about 2 hours. The preferred drying conditions facilitate controlled moisture removal from the gelatin coating while helping to maintain coating integrity, uniformity, and film structure.

At step 206, the gelatin-coated PLGA films undergo a crosslinking process using a crosslinking agent. In an embodiment, the crosslinking agent includes, but not limited to, glutaraldehyde, genipin, formaldehyde, carbodiimide crosslinkers, microbial transglutaminase, tannic acid, citric acid, oxidized polysaccharides, dialdehyde starch, riboflavin-mediated photochemical crosslinking agents, and combinations thereof. In one embodiment, the carbodiimide crosslinkers may include, but not limited to, 1-ethyl-3-(3-dimethylaminopropyl) carbodiimide (EDC) or 1-ethyl-3-(3-dimethylaminopropyl)carbodiimide/N-hydroxysuccinimide (EDC/NHS).

In preferred embodiment, the crosslinking agent might be genipin. Genipin is a well-known crosslinking agent for gelatin and gelatin-based systems and might improve the mechanical stability of the coating while reducing susceptibility to enzymatic degradation. Further, the concentration of genipin and the extent of crosslinking might be selected to tune degradation characteristics and release behaviour of the gelatin-based coating system. The genipin might enhance the mechanical strength and slow enzymatic degradation under physiological conditions in the gelatin-coated PLGA films. In an embodiment, the gelatin-based PLGA film is treated with the crosslinking process to slow down the rapid biodegradation. In an embodiment, the crosslinking process involves a reaction mechanism that comprises the opening of a dihydropyran ring of genipin, followed by nucleophilic attack by amino groups to form stable heterocyclic crosslinked structures. The resulting network includes both intermolecular and intramolecular bridges within the gelatin matrix. The degree of crosslinking is dependent on incubation time and genipin concentration, thereby enabling tunable mechanical properties and degradation rates.

In an embodiment, the parameters during the crosslinking process are as provided below. In an embodiment, the crosslinking agent comprises a concentration in the range of about 0.001% to about 2.0% w/v. In another embodiment, the concentration of the crosslinking agent is in the range of about 0.01% to about 0.5% w/v. In another embodiment, the concentration of the crosslinking agent is in the range of about 0.05% to about 0.2% w/v. In the preferred embodiment, the concentration of the crosslinking agent might be about 0.1% w/v.

In an embodiment, the crosslinking process might be carried out for a duration of about 15 minutes to about 48 hours. In another embodiment, the crosslinking process might be carried out for a duration of about 2 hours to about 24 hours. In the preferred embodiment, the crosslinking process might be carried out for a duration in the range of about 4 hours to about 12 hours.

In an embodiment, the crosslinking process might be carried out at a temperature in the range of about 20 °C to about 40 °C. In another embodiment, the crosslinking process might be carried out at a temperature in the range of about 25 °C to about 37 °C. In the preferred embodiment, the crosslinking process might be carried out at a temperature of about 37 °C.

At step 208, the crosslinked gelatin-coated PLGA films are rinsed with phosphate-buffered saline (PBS) to remove residual crosslinking agent. Further, the crosslinked gelatin-coated PLGA films are stored in a hydrated state at about 4 °C. The rinsing helps to remove residual crosslinking agent and stabilize the hydrated gelatin-coated PLGA films before drug loading and assembly.

At step 210, a specialized pro-resolving mediator (SPM) is loaded between the gelatin-coated PLGA films. In an embodiment, the RvD1 is dissolved in a pharmaceutically acceptable solvent to form an RvD1 solution. The pharmaceutically acceptable solvent might include, but not limited to, ethanol, methanol, isopropanol, dimethyl sulfoxide, acetonitrile, polyethylene glycol, propylene glycol, glycerol, phosphate-buffered saline containing a solubilizing agent, an albumin-containing solution, a cyclodextrin-containing solution, a lipid-based carrier, or combinations thereof. In a preferred embodiment, the RvD1 is dissolved in ethanol. In the preferred embodiment, the pharmaceutically acceptable solvent is ethanol. In an embodiment, the RvD1 solution is prepared by dissolving about 100 μg of Resolvin D1 (RvD1) in about 1 mL of ethanol.

In an embodiment, the RvD1 solution might have a concentration in the range of about 1 μg/mL to about 1,000 μg/mL. In another embodiment, the RvD1 solution concentration might be in the range of about 10 μg/mL to about 250 μg/mL. In the preferred embodiment, the RvD1 solution concentration might be about 100 μg/mL.

In an embodiment, the RvD1 loaded per gelatin-coated PLGA film is in the range of about 1 ng to about 10 μg. In another embodiment, the RvD1 loaded per gelatin-coated PLGA film is in the range of about 10 ng to about 1 μg. In the preferred embodiment, the amount of RvD1 loaded per gelatin-coated PLGA film might be about 100 ng. The preferred RvD1 concentration and loading amount might be adjusted depending on the desired release profile, therapeutic effect, and coating characteristics of the interface.

At step 212, the SPM-loaded gelatin-coated PLGA films are assembled into a multilayered structure to obtain the multilayered biodegradable drug delivery device. The multilayered biodegradable drug delivery prepared using method 200 provides effective and sustainable drug release at the targeted site. Further, the gelatin coating process of the proposed technology is different from conventional PLGA coating or biomaterial surface treatment methods in several respects.

In conventional PLGA-based drug systems, the drug is commonly dispersed throughout a bulk PLGA matrix or encapsulated within PLGA particles. Such approaches might expose sensitive bioactive molecule to organic solvents during fabrication and might further result in undesirable burst release, non-directional release, reduced bioactivity, or limited control over degradation characteristics.

In contrast, the proposed technology employs the gelatin layer as a functional and tunable diffusion-control layer within the multilayer biodegradable drug delivery device, which might be a passive surface coating. The gelatin coating process differs from conventional PLGA film coating methods because the gelatin layer is not merely applied as a passive surface coating or adhesion layer. Instead, the gelatin layer is incorporated as a functional, tunable diffusion-control layer within a multilayer biodegradable delivery device. The gelatin coating is applied under controlled temperature, pH, degassing, drying, and crosslinking conditions so that the gelatin layer modifies hydration, degradation, mechanical flexibility, and directional release of the SPM.

The application of the gelatin under controlled temperature, humidity, pH, and crosslinking conditions is expected to significantly influence the RvD1 release profile. In an embodiment, the thicker or more highly crosslinked gelatin coatings slow the release of RvD1 by reducing water penetration and diffusion, while the thinner or less crosslinked coatings increase diffusion and might cause faster or burst release of RvD1.

In an embodiment, the gelatin solution is maintained at about 37 °C during coating to maintain the solution in a homogeneous and fluid state. The gelatin solution is preferably adjusted to a pH of about 7.2 to about 7.4 to maintain physiologically compatible conditions and promote consistent crosslinking. The coated films are dried at about 20 °C to about 25 °C and about 30% to about 50% relative humidity for about 30 minutes to about 2 hours. The gelatin layer is subsequently crosslinked with about 0.1% w/v crosslinking agent at about 37 °C for about 4 to about 12 hours. These conditions produce a crosslinked gelatin network that slows gelatin dissolution, reduces burst release, and supports sustained RvD1 release from the multilayer film.

In an embodiment, the release profile might be tuned by adjusting at least four parameters: gelatin concentration, gelatin coating thickness, crosslinking agent concentration, and crosslinking time. For example, increasing the gelatin coating thickness from about 5 μm to about 50 μm, increasing the crosslinking agent concentration from about 0.01% to about 0.5% w/v, or extending the crosslinking time from about 2 hours to about 24 hours is expected to slow RvD1 release.

### In Vitro Biological Assays

Primary cell cultures of endothelial cells (ECs) and vascular smooth muscle cells (VSMCs) were obtained from human greater saphenous veins that were discarded during bypass surgeries for biological evaluation (Elzoghby & Elgindy, 2020). The drug delivery device containing Resolvin D1 (RvD1) or an ethanol control were positioned in transwell inserts above the cells to investigate the biological effects of RvD1 (Elzoghby & Elgindy, 2020). For the nuclear factor-κB (NF-κB) assay, VSMCs were treated with the drug delivery device for 18 hours and then stimulated with TNF-α. After treatment, the cells were fixed and stained with a fluorescent dye to visualize the nuclear translocation of NF-κB using a fluorescent microscope (Machein & Plate, 2019). In the cell proliferation assay, VSMCs were serum-starved and stimulated with media. After exposure to the drug delivery device, cell counts were conducted to evaluate the impact of RvD1 on proliferation (Elzoghby & Elgindy, 2020). Migration assays involved creating a wound in a confluent VSMC monolayer, with the drug delivery device loaded with RvD1 solution were placed in the wells to assess changes in cell migration over 24 hours; photographs of the wounds were taken at regular intervals to quantify migration (Machein & Plate, 2019). Lastly, cells were exposed to a vehicle device (which is conventionally known) and the drug delivery device of the claimed proposed technology) to evaluate cytotoxicity, and cell viability was measured using an MTT assay, which quantifies viable cells based on their metabolic activity.

The vehicle device is a control device having substantially the same layers as the multilayered as the RvD1-loaded drug delivery device, but without RvD1. In one embodiment, the vehicle device comprises the same first layer, intermediate layer, and second layer configuration and is treated with the same solvent or carrier used for RvD1 loading, such as ethanol, without inclusion of RvD1. The vehicle device controls for the effects of the polymeric layers, gelatin content, device structure, and loading solvent, whereas the multilayered biodegradable drug delivery device comprises RvD1 loaded between adjacent layers for localized and sustained release to a targeted vascular site.

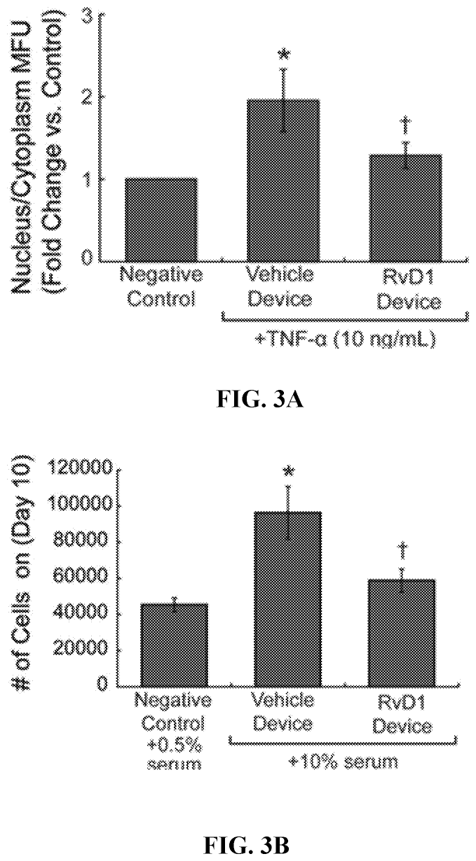

The FIGS. 3A to 3C are the experimental results to illustrate the biological activity of RvD1 released from the drug delivery device using vascular smooth muscle cells. The experiments involve vascular smooth muscle cells exposed to the RvD1-loaded multilayered biodegradable drug delivery device, cells exposed to a vehicle device, and negative control cells. The vehicle device had the same multilayer PLGA/gelatin configuration as the RvD1-loaded device, but did not contain active RvD1. Instead, the vehicle device contained the solvent or carrier used for loading RvD1, such as ethanol, without RvD1. The negative control refers to cells maintained without exposure to the RvD1-loaded device. In the nuclear factor-κB (NF-κB) assay, the negative control might include cells not stimulated with TNF-α. The TNF-α might be Tumor Necrosis Factor-alpha (TNF-α).

Referring to FIG. 3A, the effect of the drug delivery device with loaded RvD1 solution on TNF-α-induced NF-κB activation in vascular smooth muscle cells is evaluated. NF-κB activation was assessed by measuring nuclear translocation of the p65 subunit. Compared with the vehicle device, the drug delivery device with loaded RvD1 solution reduced nuclear p 65 translocation by about 36%, indicating that RvD1 released from the device inhibited inflammatory NF-κB signaling.

Referring to FIG. 3B, the effect of the drug delivery device with loaded RvD1 solution on vascular smooth muscle cell proliferation is evaluated. Cells exposed to the RvD1-loaded device showed about a 39% reduction in proliferation compared with the vehicle device, indicating that RvD1 released from the drug delivery device with loaded RvD1 solution reduced vascular smooth muscle cell growth.

Referring to FIG. 3C, the effect of the drug delivery device with loaded RvD1 solution on vascular smooth muscle cell migration is evaluated. In the migration assay, a wound or gap was created in a confluent vascular smooth muscle cell monolayer, and the extent of wound closure was monitored over time. Cells exposed to the drug delivery device with loaded RvD1 solution showed about a 22% reduction in migration compared with the vehicle device, indicating that RvD1 released from the device reduced vascular smooth muscle cell motility.

FIGS. 3A, 3B, and 3C demonstrate that the drug delivery device with loaded RvD1 solution retains biological activity after loading and release. The released RvD1 suppresses inflammatory signalling, reduces vascular smooth muscle cell proliferation, and reduces vascular smooth muscle cell migration, supporting the use of the drug delivery device for localized treatment of vascular inflammation, vascular injury, graft failure, restenosis, thrombosis, and related vascular conditions.

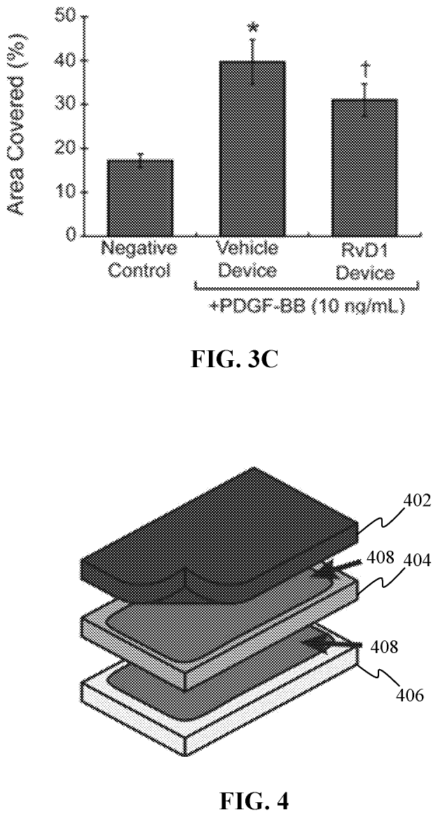

Referring to FIG. 4, in an embodiment, the multilayered biodegradable drug delivery device is a tri-layered structure. The tri-layered structure comprises a first layer 402, a second layer 406, and an intermediate layer 404 disposed between the first layer 402 and the second layer 406. In one embodiment, the first layer 402 has 70-95% PLGA and 5-30% gelatin; the intermediate layer 404 has 55-85% PLGA and 15-45% gelatin; and the second layer 406 has 25-70% PLGA and 30-75% gelatin. In the preferred embodiment, the first layer 402 has 85% PLGA and 15% gelatin; the intermediate layer 404 has 75% PLGA and 25% gelatin; and the second layer having 50% PLGA and 50% gelatin. Further, the tri-layered structure comprises a bioactive agent 408 that includes SPM. The SPM comprises dissolved Resolvin D1 (RvD1) in ethanol to obtain an RvD1 solution. In an embodiment, about 100 ng of RvD1 solution is loaded between the first layer 402 and the intermediate layer 404, and about 100 ng of RvD1 solution is loaded between the intermediate layer 404 and the second layer 406, allowing the release of RvD1 to the targeted site. The targeted site is a site of a vascular condition selected from the group consisting of vascular injury, vascular inflammation, ischemia-reperfusion injury, endotoxin-induced vascular damage, vascular trauma, graft failure, and thrombosis. In an embodiment, the device is attached to a blood vessel of a subject. The subject might be human. In an embodiment, the first layer of the device is towards an outer surface of the blood vessel, and the second layer is towards an inner surface of the blood vessel.

The multilayer structure comprises layers having different PLGA-to-gelatin ratios, including but not limited to about 85:15, about 75:25, and about 50:50 PLGA/gelatin compositions. The use of differing layer compositions might generate asymmetric hydration and degradation behavior across the multilayer structure, thereby biasing release of the RvD1 toward the vascular injury site or tissue-facing surface.

In certain embodiments, the gelatin coating is crosslinked using the crosslinking agent. The degree of crosslinking might be adjusted to tune degradation rate, swelling behavior, mechanical properties, and diffusion of the RvD1 through the gelatin matrix. The resulting multilayer construct might thereby provide a conformable, biodegradable, vascular-facing delivery device capable of localized and sustained release of RvD1.

In an embodiment, the disclosed approach enables targeted and sustained delivery of Resolvin D1 (RvD1) directly to sites of vascular injury. This localized administration maximizes the therapeutic efficacy of RvD1 at the site of damage while substantially reducing systemic exposure and limiting potential effects on adjacent healthy vascular tissue.

The drug delivery device of the proposed technology is capable of delivering therapeutically effective concentrations of the Resolvin D1 (RvD1) to vascular tissues in both ex vivo and in vivo experimental models. Its flexible and conformable structure enables direct attachment to blood vessels, rendering it suitable for the treatment of various acute and sub-acute inflammatory conditions, including ischemia-reperfusion injury, endotoxin-induced vascular damage, and other forms of vascular trauma. This biodegradable drug delivery device offers considerable therapeutic promise for a broad spectrum of surgical procedures, where localized, sustained release of pro-resolving lipid mediators such as RvD1 might promote tissue repair and inflammation resolution.

The processes described above is described as a sequence of steps, this was done solely for the sake of illustration. Accordingly, it is contemplated that some steps might be added, some steps might be omitted, the order of the steps might be re-arranged, or some steps might be performed simultaneously.

Although embodiments have been described with reference to specific example embodiments, it will be evident that various modifications and changes might be made to these embodiments without departing from the broader spirit and scope of the system and process or method described herein. Accordingly, the specification and drawings are to be regarded in an illustrative rather than a restrictive sense.

Many alterations and modifications of the present invention will no doubt become apparent to a person of ordinary skill in the art after having read the foregoing description. It is to be understood that the phraseology or terminology employed herein is for the purpose of description and not of limitation. It is to be understood that the description above contains many specifications, these should not be construed as limiting the scope of the invention but as merely providing illustrations of some of the personally preferred embodiments of this invention.

What is claimed is:

1. A method of preparing a multilayered biodegradable drug delivery device, the method comprising:
preparing a plurality of poly(lactic-co-glycolic acid) (PLGA) films;
coating each PLGA film with a gelatin solution to obtain gelatin-coated PLGA films;
crosslinking the gelatin-coated PLGA films using a crosslinking agent;
loading a specialized pro-resolving mediator (SPM) between the gelatin-coated PLGA films; and
assembling the SPM-loaded gelatin-coated PLGA films into a multilayered structure to obtain the multilayered biodegradable drug delivery device.

2. The method of claim 1, wherein preparing a plurality of poly(lactic-co-glycolic acid) (PLGA) films comprises:
dissolving poly(lactic-co-glycolic acid) in 2,2,2-trifluoroethanol (TFE) to obtain a PLGA solution;
spin-casting the PLGA solution onto polydimethylsiloxane (PDMS) substrates to form PLGA films; and
drying the PLGA films to obtain discrete PLGA films.

3. The method of claim 2, wherein preparing the PLGA solution comprises dissolving poly(lactic-co-glycolic acid) in 2,2,2-trifluoroethanol at a concentration of about 50 mg/mL to about 500 mg/mL.

4. The method of claim 3, wherein preparing the PLGA solution comprises dissolving poly(lactic-co-glycolic acid) in 2,2,2-trifluoroethanol at a concentration of about 300 mg/mL.

5. The method of claim 1, wherein the gelatin solution is prepared by:
dissolving a gelatin powder in an aqueous medium at a temperature in the range of about 30 °C to about 40 °C under continuous stirring at about 200 rpm to about 400 rpm for about 30 minutes to about 90 minutes, and maintained at a pH of 4.0 to 9.0 to form the gelatin solution; and
degassing the gelatin solution under vacuum for about 3 minutes to about 10 minutes.

6. The method of claim 5, wherein the aqueous medium is distilled water or a phosphate-buffered saline (PBS) solution.

7. The method of claim 5, wherein the concentration of the gelatin in the gelatin solution is in the range of about 1% (w/v) to about 5% (w/v).

8. The method of claim 7, wherein the concentration of the gelatin in the gelatin solution is about 2% (w/v).

9. The method of claim 5, wherein the degassed gelatin solution is maintained at a temperature of about 37 °C for about 30 seconds to about 10 minutes to form a uniform thickness of gelatin coating on the PLGA film, wherein the degassing of the gelatin solution is performed for about 5 minutes.

10. The method of claim 9, wherein the thickness of gelatin coating is in the range of 1 μm to about 100 μm on each PLGA film.

11. The method of claim 10, wherein the thickness of gelatin coating is about 10 μm on each PLGA film.

12. The method of claim 1, wherein the crosslinking agent is any one of glutaraldehyde, genipin, formaldehyde, carbodiimide crosslinkers, microbial transglutaminase, tannic acid, citric acid, oxidized polysaccharides, dialdehyde starch, riboflavin-mediated photochemical crosslinking agents, or combinations thereof.

13. The method of claim 12, wherein the crosslinking agent comprises a concentration in the range of about 0.001% to about 2.0% w/v, wherein the concentration of the crosslinking agent is about 0.1% w/v.

14. The method of claim 1, comprising rinsing the crosslinked gelatin-coated PLGA films with phosphate-buffered saline (PBS) to remove residual crosslinking agent, and storing the crosslinked gelatin-coated PLGA films in a hydrated state at about 4 °C.

15. The method of claim 1, wherein the SPM comprises dissolved Resolvin D1 (RvD1) in ethanol to form an RvD1 solution.

16. The method of claim 1, wherein the multilayered biodegradable drug delivery device is a tri-layered structure, wherein the tri-layered structure comprises a first layer having 70-95% PLGA and 5-30% gelatin; an intermediate layer having 55-85% PLGA and 15-45% gelatin; a second layer having 25-70% PLGA and 30-75% gelatin.

17. The method of claim 16, wherein the tri-layered structure further comprises a bioactive agent that includes SPM, wherein about 1 ng to about 10 μg of SPM is loaded between the first layer and the intermediate layer, and about 1 ng to about 10 μg of SPM is loaded between the intermediate layer and the second layer, allowing the release of SPM having the Resolvin D1 (RvD1) at a targeted site.
